(19) United States
(12) Patent Application Publication
(10) Pub. No.: US 2015/0359567 A1
(43) Pub. Date: Dec. 17, 2015

(54) EXFOLIATING MITT/MITTEN

(71) Applicant: Margarita Vassileva, Savannah, GA (US)

(72) Inventor: Margarita Vassileva, Savannah, GA (US)

(21) Appl. No.: 14/735,980

(22) Filed: Jun. 10, 2015

Related U.S. Application Data
(60) Provisional application No. 62/013,166, filed on Jun. 17, 2014.

(51) Int. Cl. A61B 17/54; A47K 7/02

(52) U.S. Cl. CPC A61B 17/54; A47K 7/02

(57) ABSTRACT

Exfoliation involves the removal of the oldest dead skin cells on the skin's outermost surface. It can be achieved through mechanical or chemical means. The mechanical process involves physically scrubbing the skin with abrasive exfoliates. It has been discovered that a wetted panel of silk material exhibits abrasive qualities and may be used as a hand-held product for exfoliating skin.

### CROSS-REFERENCES TO RELATED APPLICATIONS

This application claims the benefit of U.S. Provisional Patent Application No. 62/013,166, filed Jun. 17, 2014, the entire content of which is herein incorporated by reference.

### STATEMENT REGARDING FEDERALLY SPONSORED RESEARCH OR DEVELOPMENT

(NOT APPLICABLE)

### BACKGROUND OF THE INVENTION

There are existing products that claim they will exfoliate skin. One example is a multi-layer washcloth where silk may be used as an inner layer. The accent is on the outer layer that is made out of nylon net material. Other examples include an article composed of water soluble fibers and comprising exfoliate particles, bilayer articles with two sides—rough and smooth, where the use of some natural fibers including silk is mentioned in the absorbent layer, and a substrate laden with a fluid cosmetic composition. None of the existing products appreciates the use of wetted silk as an exfoliating material.

### BRIEF SUMMARY OF THE INVENTION

It has been discovered that wet silk has abrasive qualities and is effective for exfoliating skin.

In an exemplary embodiment, a method of exfoliating skin from a skin surface includes the steps of wetting a silk panel, and scrubbing the skin surface with the wet silk panel. The silk panel may be wetted with water. The method may further include, prior to the wetting step, a step of providing a silk panel consisting of silk. The method may still further include shaping the silk panel into a mitt or mitten. The shaping step may be practiced by aligning two silk panels, and sewing the two silk panels together along an outside edge except for an open section sized to receive a user's hand or a tool.

In another exemplary embodiment, a hand-held product for exfoliating skin includes a main panel of material that consists of silk, where the main panel is shaped to facilitate manipulation by hand. The material may be altered such that the material exhibits abrasive qualities. The material may be altered by being wetted. The altered material may be weakened by being altered.

### BRIEF DESCRIPTION OF THE DRAWINGS

These and other aspects and advantages will be described in detail with reference to the accompanying drawings, in which:

FIG. 1 shows an exemplary mitt/mitten for exfoliating skin;

FIG. 2 shows a basic construction using silk panels; and

FIG. 3 is an exemplary illustration of an application for exfoliating skin using the product of the described embodiments.

### DETAILED DESCRIPTION OF THE INVENTION

Exfoliation involves the removal of the oldest dead skin cells on the skin's outermost surface. It can be achieved through mechanical or chemical means. The mechanical process involves physically scrubbing the skin with abrasive exfoliants. It has been discovered that wet silk exhibits abrasive qualities. It is well-known that silk is a very soft material when dry. When wet, however, the abrasive qualities of the material are particularly suited for exfoliating skin. Filaments of silk material are generally strong due to the linear, beta-configuration polymers and a very crystalline polymer system. These factors permit many more hydrogen bonds to be formed in a much more regular manner. When wet, silk loses strength. This is due to water molecules hydrolyzing a significant number of hydrogen bonds and in the process weakening the silk polymer. This weakening, it has been discovered, gives the silk material its abrasive qualities. The material is thus altered, i.e., weakened, by being wetted.

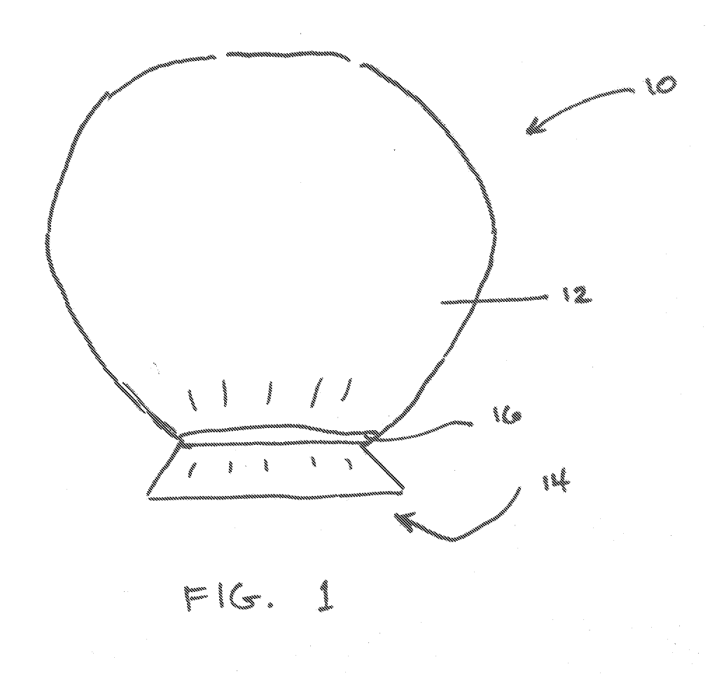

With reference to FIG. 1, an exfoliating mitt or mitten 10 is formed using a silk material. The silk material may be processed silk or raw silk. Exemplary processed silk includes Charmeuse, Chiffon, Crepe de Chine, Habutai etc. The mitt or mitten 10 may be assembled in any configuration, and the mitt shown in FIG. 1 is exemplary. The mitt or mitten 10 includes two panels of silk material 12 cut and sewn together preferably into a shape and construction that facilitates manipulation by hand. An opening 14 is typically provided in a mitt or mitten, and an elastic material 16 serves to cinch the end to help secure the mitt or mitten 10 on a user's hand.

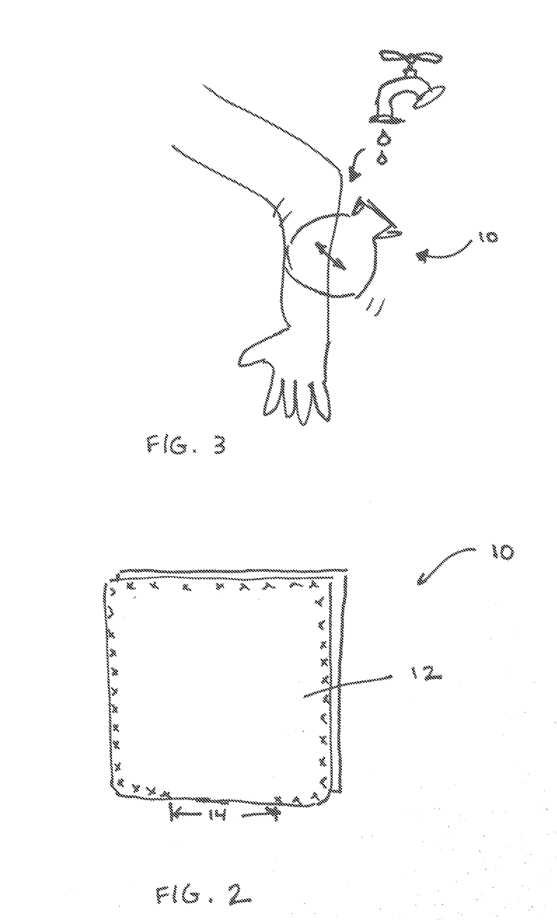

FIG. 2 shows a basic construction where two silk panels 12 are aligned with each other, and the silk panels 12 are sewn together along an outside edge except for an open section 14 sized to receive a user's hand or a tool. The silk material may also form part of an exfoliating brush or the like where a silk disc or alternatively shaped silk swath is secured to a brush.

The mitt 10 is all natural (silk is a natural protein fiber) with a simple design and function. In use, there is no need for abrasive dry particles or chemical liquids—just water. With reference to FIG. 3, in a method of exfoliating skin from a skin surface, a silk panel in the form of a mitt or mitten 10 or other form is wetted, preferably with water. Subsequently, the skin surface is scrubbed with the wet silk panel. The skin may be scrubbed in various directions, but it is preferably scrubbed in varying directions such as up/down, side/side, and in a circular motion.

Dead skin cells and dirt can be readily seen after exfoliating with the silk mitt. Typical products that claim to be suitable for exfoliating skin do not function as efficiently and often lack any physical evidence to prove functionality. The mitt according to the described embodiments provides superior functionality in a simple configuration.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

1. A method of exfoliating skin from a skin surface, the method comprising:
wetting a silk panel; and
scrubbing the skin surface with the wet silk panel.

2. A method according to claim 1, wherein the silk panel is wetted with water.

3. A method according to claim 2, further comprising, prior to the wetting step, providing a silk panel consisting of silk.

4. A method according to claim 3, further comprising, shaping the silk panel into a mitt or mitten.

5. A method according to claim 4, wherein the shaping step is practiced by aligning two silk panels, and sewing the two silk panels together along an outside edge except for an open section sized to receive a user's hand or a tool.

6. A hand-held product for exfoliating skin, comprising a main panel of material, wherein the material consists of silk, and wherein the main panel is shaped to facilitate manipulation by hand.

7. A hand-held product for exfoliating skin according to claim 6, wherein the material is altered such that the material exhibits abrasive qualities.

8. A hand-held product for exfoliating skin according to claim 7, wherein the material is altered by being wetted.

9. A hand-held product for exfoliating skin according to claim 7, wherein the altered material is weakened by being altered.
